(19) United States
(12) Patent Application Publication
(10) Pub. No.: US 2024/0061382 A1
(43) Pub. Date: Feb. 22, 2024

(54) SURFACE MODIFICATION BASED ON DETERMINED CONDITIONS

(71) Applicant: Praesidium, Inc., St. George, UT (US)

(72) Inventors: Seth Campbell, Washington, UT (US); Gian Franco Sacco, Pasadena, CA (US)

(73) Assignee: Praesidium, Inc., St. George, UT (US)

(21) Appl. No.: 17/891,715

(22) Filed: Aug. 19, 2022

(51) Int. Cl. G05B 13/02

(52) U.S. Cl. CPC G05B 13/027; A61B 5/6892

(57) ABSTRACT

An example operation includes one or more of receiving data from a sensor related to a user occupying a surface, responsive to the received data, detecting a condition above a threshold of the user, responsive to the detected condition above the threshold, modifying at least one compartment of the surface, comparing a current condition of the user to the detected condition, and responsive to the current condition below the threshold, setting the modified at least one compartment of the surface at a fixed level.

### BACKGROUND

The present document generally pertains to a modification of a surface and, more particularly but not by way of limitation, to alter the surface based on conditions of an object on the surface.

### SUMMARY

One example embodiment provides a method that includes one or more of receiving data from a sensor related to a user occupying a surface, responsive to the received data, detecting a condition above a threshold of the user, responsive to the detected condition above the threshold, modifying at least one compartment of the surface, comparing a current condition of the user to the detected condition, and responsive to the current condition below the threshold, setting the modified at least one compartment of the surface at a fixed level.

Another example embodiment provides a system that includes a memory communicably coupled to a processor, wherein the processor performs one or more of receive data from a sensor related to a user that occupies a surface, responsive to the received data, detect a condition above a threshold of the user, responsive to the detected condition above the threshold, modify at least one compartment of the surface, compare a current condition of the user to the detected condition, and responsive to the current condition below the threshold, set the modified at least one compartment of the surface at a fixed level.

A further example embodiment provides a computer readable storage medium comprising instructions, that when read by a processor, cause the processor to perform one or more of receiving data from a sensor related to a user occupying a surface, responsive to the received data, detecting a condition above a threshold of the user, responsive to the detected condition above the threshold, modifying at least one compartment of the surface, comparing a current condition of the user to the detected condition, and responsive to the current condition below the threshold, setting the modified at least one compartment of the surface at a fixed level.

### BRIEF DESCRIPTION OF THE DRAWINGS

FIG. 1 illustrates an example system, according to example embodiments.

FIG. 2 illustrates an example flowchart, according to example embodiments.

FIG. 3 illustrates a flow diagram according to example embodiments.

FIG. 4 illustrates another flow diagram, according to example embodiments.

FIG. 5 illustrates an example system that supports one or more example embodiments.

### DETAILED DESCRIPTION

It will be readily understood that the instant components, as generally described and illustrated in the figures herein, may be arranged and designed in a wide variety of different configurations. Thus, the following detailed description of the embodiments of at least one of a method, apparatus, computer readable storage medium, and system, as represented in the attached figures, is not intended to limit the scope of the application as claimed but is merely representative of selected embodiments. Multiple embodiments depicted herein are not intended to limit the scope of the solution. The computer-readable storage medium may be a non-transitory computer readable medium or a non-transitory computer readable storage medium.

Communications between the surface (such as a mattress, a seat, etc.) and certain entities, such as remote servers, other surfaces, and local computing devices (e.g., smartphones, personal computers, transport-embedded computers, etc.) may be sent and/or received and processed by one or more ‘components’ which may be hardware, firmware, software or a combination thereof. The components may be part of any of these entities or computing devices or certain other computing devices. In one example, consensus decisions related to blockchain transactions may be performed by one or more computing devices or components (which may be any element described and/or depicted herein) associated with the surface and one or more of the components outside or at a remote location from the surface.

The instant features, structures, or characteristics described in this specification may be combined in any suitable manner in one or more embodiments. For example, the usage of the phrases “example embodiments,” “some embodiments,” or another similar language throughout this specification refers to the fact that a particular feature, structure, or characteristic described in connection with the embodiment may be included in at least one example. Thus, appearances of the phrases “example embodiments,” “in some embodiments,” “in other embodiments,” or other similar language throughout this specification do not necessarily all refer to the same group of embodiments, and the described features, structures, or characteristics may be combined in any suitable manner in one or more embodiments. In the diagrams, any connection between elements can permit one-way and/or two-way communication, even if the depicted connection is a one-way or two-way arrow.

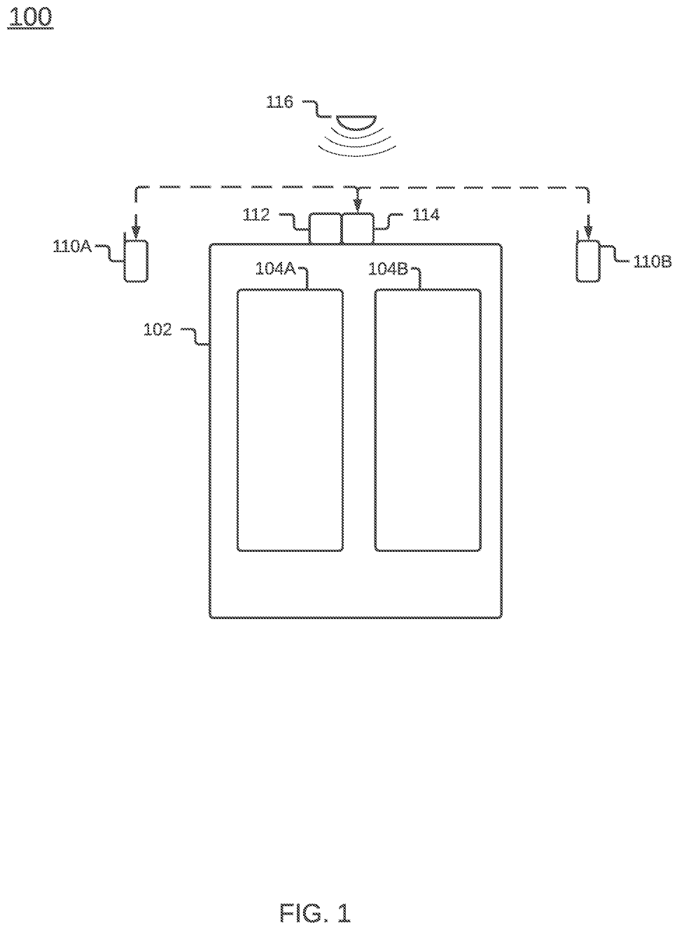

FIG. 1 shows a system 100, according to example embodiments. A surface (e.g., a mattress, a blanket, a floor, a seat cushion, a sofa cushion, a car seat, or the like) 102 is shown, containing two compartments, 104A/104B. One or more compartments in various sizes, shapes, and positions may be integrated into or on the surface. The surface may be permanently attached to another object (such as a base) or may be detachably attached (removable) from a base. The compartments may be filled with one or more substances, such as liquid, air, or any other substance. The compartments may be inflated or deflated, wherein the substance is modified by a device, such as mechanism 112 which moderates an amount of the substance that flows into and out of the compartments. The mechanism 112 may be triggered to add the substance to one or more of the left compartment 104A and the right compartment 104B. The mechanism 112 may also be triggered to remove the substance from one or more of the left or right compartments. A communication device 114 is communicably coupled to the mechanism 112 and is configured to send and receive messages to and from connected devices 110A/110B that may be associated with one or more users of the surface. The devices 110A/110B may be associated with one or more users of the surface 102. For example, the left device 110A is associated with the user that occupies the left side of the surface 102, and the right device 110B is associated with the user that occupies the right side of the surface 102. The implementation of the functionality described herein may entirely or partially reside in the surface 102, the compartments 104A/104B, the devices 110A/110B, the mechanism 112, the communication device 114, and/or the sensor 116. In another embodiment, the current solution may fully or partially reside in a server/computer communicably coupled to the system 100, wherein messages between the system 100 and the server/computer route through a network.

The system 100 may be modified by one or more devices 110A/110B associated with one or more users of the surface 102. The devices 110A/110B may be mobile phones, tablets, laptops, computers, watches, or devices containing a memory and processor. The current solution executing on at least one device, 110A, and 110B, communicates with the communication device 114 on or proximate the surface 102. The communication device 114 may communicate with the devices associated with the users through common wireless means, such as Bluetooth, Wi-Fi, and the like.

A sensor 116 is present in the system 100 and may be a radar device (such as a multimeter wave radar), a pressure detection device, a motion detection device, a microphone device, and any other type of sensor and may be mounted on or proximate the surface 102, in the surface 102, or any other location proximate the surface 102. The sensor communicates with the surface 102 through the communication device 114 via a wired or wireless connection. The communication device 114 receives data from the sensor 116. In one embodiment, the sensor is in the devices 110A/110B. The sensor determines conditions of the one or more users on the surface 102. All of the devices may be omnidirectional or multidirectional, such as each sensor can receive data and/or send data.

The user may interact with the surface 102 through an application on the device 110A/110B, wherein the application may execute the current solution. The application may contain a Graphical User Interface (GUI), allowing users to modify one or more of the compartments 104A/104B in or on the surface 102. In one embodiment, the GUI may also allow the selection of adjustments to one or more of the sensor 116, the surface 102, the compartments 104A/104B, and may set any of these elements of the system 100 to a new setting or a previously saved setting. The GUI may also all the setting selection to the more than one threshold level. The GUI may also allow the selection of what is desired to be measured, such as heart rate, blood pressure, amount and number of movements, a level of audio of sounds such as snoring, gasping, etc.

One or more of the compartments 104A/104B in one embodiment are dynamically adjusted to alleviate a condition. The mechanism 112 receives data from the sensor 116 and responds by altering one or more elements of the system 100, such as an inclination or declination of a frame holding the surface 102, one or more of the compartments 104A/104B (for example, inflating, deflating, injecting the substance, removing the substance, cooling, heating, vibrating, etc.), and/or any other element of the system, to alleviate the condition. In one embodiment, the substance in the compartments 104A/104B may be heated or cooled. This is accomplished by a heating or cooling element in a device, such as in the mechanism 112. The condition may be snoring, a change in breathing, a change in heart rate, an indication of a numb arm, constant body movement, or any other condition or body position/movement. The conditions, which may be determined by the current application by analysis of data from the sensor 116, may include extensive movement of the user, tossing and turning of the user, readjustment of the user, and sounds of the user, including such sounds as gasping for air, snoring, coughing, and the like. These conditions, and similar conditions may be determined by the instant solution executing on a processor, such as a processor in the mechanism 112, and may utilize algorithms commonly used in data analysis of objects, such as object detection. Object detection algorithms may be used to determine irregularities. Through deep learning, the current application can determine what the object is. Object detection algorithms exist and are utilized by the current application, in one embodiment, to perform object detection for objects one or near the surface 102. There are multiple algorithms available for object detection. For example, the following list describes possible algorithms used to perform object detection. Other algorithms in the object detection field are possible as well. The current application may use Convolutional Neural Networks (CNN). In CNN, an image (which may include a portion of a video wherein the video is split into different images) is passed to a processor and analyzed. The vehicle processor or a processor off-board the vehicle may perform the analysis off-board. The image is sent through various convolutions and pooling layers in the analysis, and a corresponding object class is returned for each image input. The corresponding class includes a string for what the object is and a percentage of validation of what the object is. Other object detection algorithms exist and may be utilized by the current processor to perform object detection, including (but not limited to) Region-Based Convolutional Neural Network (RCNN) that uses selective searching to generate regions from each image, Fast RCNN, where each image is passed only once to the CNN and feature maps are extracted, and selective search is used on these maps to generate predictions and Faster RCNN, which replaces the selective search method with region proposal networks which make the algorithm much faster.

In one embodiment, a device dynamically plays a sound to aid in the sleep of the one or more users. The device be a mobile device of a user of the surface, a speaker communicably coupled to the mechanism 112, or any other device that may transmit and/or amplify sounds. For example, a sound of waves may be played. The sound may be played when the mechanism 112 alters the compartments 104A/104B to mask the noise. In one embodiment, when the system 100 determines the person is moving, the sound may be modified (either at a higher or lower volume) to aid the user to return to sleep or wake from sleep.

In one embodiment, the user is associated with a portion or side of the surface 102 by the system 100. When more than one person is on the surface 102, the system 100 determines which user is on which side of the surface 102.

In one embodiment, a side of the surface 102 associated to a particular user by the determination of a proximity of a mobile device associated with each respective user. The processor of the mobile device sends a message containing details of the user and coordinates of the mobile device 110A/110B to the system 100, such as to the processor of one or more of the communication device 114 and the mechanism 112. The device in the system 100 uses these coordinates to determine which side of the surface 102 is associated with each user. For example, User-A, on the left side of the surface 102 has in possession or proximate User-A a mobile device 110A (such as on a nightstand near the surface). The system 100 determines that User-A is associated with the left side of the surface 102. In one embodiment, a setup process of the instant solution associates a user with a side of the surface 102 where a given user is asked to sit or lie on the side he/she wants to occupy and by detecting a presence of the user, the instant solution associates that side with that user. When the surface 102 is occupied by a user, the sensor 116 (which may be a pressure sensor in the surface 102), may detect the user and associate that side of the surface with that user. For example, the user, in the setup process, may provide details to an application executing on a device, such as a mobile device. The details may be one or more of a name, a weight, an age, and the like. When the user occupies the surface, the side of the surface may be associated by the instant solution to match the user that provided the details.

In another embodiment, the user performs a scan using a mobile device 110A/110B of least one code on the surface 102, such as a scan of a QR code, a bar code, or any other type of information that can be analyzed by the device, which may be used by the system 100 to determine a side of the surface 102 that is associated with a user. In one embodiment, other technology may be used to ascertain a side of the surface 102 with a user, such as wireless communication (such as Bluetooth) where a protocol stack is present in both the mobile device 110A/110B and the surface 102. In one embodiment, a special passcode may be presented on a GUI on a mobile device 110A/110B, wherein the passcode is entered and thereby associated the user of the mobile device 110A/110B with the side of the surface 102 associated with the entered passcode.

The at least one QR code will be on the surface 102 near the portion of the surface 102 where the user will occupy the surface 102. There may be two different QR codes on the surface 102, such that a left QR code is associated with the left portion of the surface 102, and the right QR code is associated with the right portion of the surface 102. Therefore, the left compartment 104A is associated with the user on the left portion of the surface 102, and the right compartment 104B is associated with the right portion of the surface 102. When the code is scanned, the device 110A/110B sends a notification to the communication device 114, indicating which portion of the surface 102 and thus which compartment 104A/104B is associated with that user. In another embodiment, when a user sits on the surface 102, pressure sensors on the surface 102 detect the user's presence, and the system 100 knows which user has applied pressure to the surface 102. The device 110A/110B proximate the pressure is the device associated with the user that applied the pressure to the surface 102.

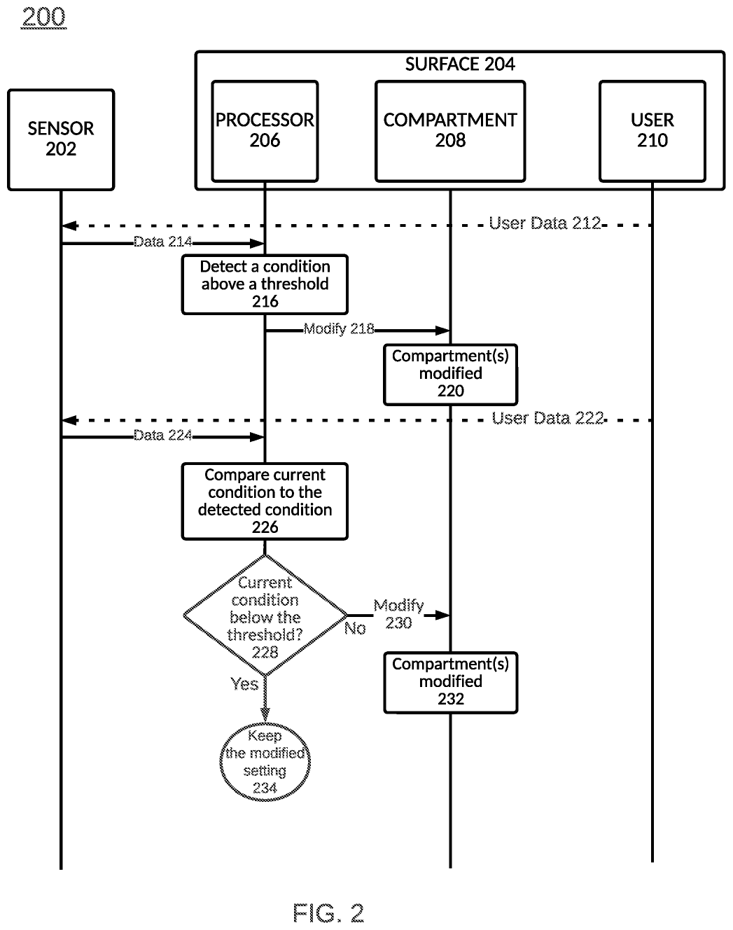

FIG. 2 shows a flowchart 200, according to example embodiments. A surface 204 is depicted, including three elements, a processor 206, one or more compartments 208, and one or more users 210. The surface 204 may be a mattress, a portion of a floor, a blanket, a covering, a seat cushion, a seat in a vehicle, and the like. The surface 204 may be communicably coupled with the processor 206. The processor may be in a device connected to the surface, a frame of the surface, or anywhere proximate the surface. The processor may receive messages (wired and wirelessly) from another processor, such as a processor coupled to a device associated with users of the surface, such as a mobile device, for example. In one embodiment, a communication device may be present where the external devices (i.e., the mobile devices) communicate with the communications device and sends messages to the processor 206. The one or more compartments 208 are in or on the surface 204. The one or more compartments may be filled with air or a substance and may be perforated, allowing for the one or more compartments to be enlarged or reduced. At least one or more users 210 may be present on the surface. A sensor 202 is present on or proximate the surface 204. The sensor may monitor the user 210 on the surface and interact with the processor 206. The processor may modify the size of the compartment 208 according to the received data from the sensor 202.

The sensor 202 receives data 212 of the surface 204 and the one or more users 210 on the surface 204. Data may be received continually or at intervals, such as every 10 seconds, every minute, every 5 minutes, etc. More than one sensor 202 may be present in the system, and the one or more sensors may be pointed towards the surface 204 to collect data therein. The sensor 202 may detect conditions of the user 210, such as the blood flow, breathing, heart rate, movement of the user, such as movement of the chest, and the like. The sensor 202 may also detect sounds, such as coughing, snoring, gasping, distress, and the like. Data received by the sensor is sent 214 to the processor 206. The data may be sent to the processor continually or at an interval, such as when the sensor is scheduled to collect the data. The current application, executing in the processor 206 analyzes the received data 214 to detect a condition below a threshold 216. The threshold may be established and hardcoded into the logic of the current application or may be set by a user or other entity, such as the user 210 of the surface 204. The threshold setting may be determined through a configuration module in the current application in one embodiment. In one embodiment, an outside entity may set the one or more thresholds of the system 200. For example, a server associated with the outside entity may communicate with one or more processors in the system 200, wherein the communication may be routed through a network. The outside entity may be associated with a doctor's office, a health provider, and the like.

The threshold may be related to an occurrence of the user's 210 condition a number of times in a period, such as 10 minutes. The condition may be one or more of an amount of movement of the user 210 or a limb of the user 210, an amount of time between breaths of the user 210, a maximum amount of sound, such as distress of the user 210, snoring, gasping, coughing, and the like. The threshold may also be associated with other characteristics of the user, such as a rate of breathing, heart rate, and the like. For example, if the user stops breathing for a period of time more often than a threshold amount, such as four times over two minutes, then the current application will act to attempt to relieve the condition. In another example, the user coughs a number of times greater than a threshold in a period, such as when the user coughs more than three times in two minutes. In yet another example, the received data detects that the legs of the user moved more than a threshold number of times, such as four times, in two minutes. In one embodiment, the instant solution may determine to make one or more adjustments of any type (inflate-deflate a compartment 208, play a sound, send a notification) as a result of analyzing multiple conditions (as further described herein) of the user, such as vitals, movement, snoring, etc., using logic, such as a Machine Learning algorithm or any other algorithm. In one embodiment, the adjustment can be in real-time as described herein or can be a value that is learned by the use of logic, such as a Reinforcement Learning algorithm or any other algorithm, over a certain period of time by the modifying of the surface for a time (such as every night) until an event, such as it is determined that optimal sleep is achieved. The instant solution may obtain a feedback through a GUI displayed on a display associated with the system or a display associated with the user, such as a mobile device. The actual optimal value can be adjusted periodically to account for changed conditions or sleeping patterns, such as due to sickness, life events, such as pregnancy, and the like.

When the detected condition occurs above the threshold 216, the current application executing on the processor 206 modifies 220 the compartment 208 by a modify command 218 sent to the compartment via the processor 206. The modification may be to the size of the compartment 208, such as increasing or decreasing the amount of substance in the compartment 208.

After the modification of the compartment 220, the sensor continues to receive data 222, wherein the data is sent to the processor 206. The current application executing on a device, such as the processor 206 associated with the surface 204, compares the current condition to the previously detected condition 226 over a period. For example, if the detected condition is a number of coughs in a period, then after the modification to the compartment 220, the current number of coughs over the period is determined. If the current conditions are below the threshold 228, then the current application executing on the processor 206 establishes the current setting as the ongoing setting (the fixed setting) for the compartment 208. If not, the compartment 208 is further modified 232 through a modify command 230 sent to the compartment via the processor 206.

In one embodiment, one or more of the modifications of the compartments 220 and/or 232 may be a micro-modification. A micro-modification may be a portion of a normal modification. For example, the compartment 208 may be adjusted a small amount, such as a 5% increase. The current application determines the outcome of the micro-modification and whether the change lowered or raised the user's condition, and future modifications are adjusted according to the determined outcome.

In one embodiment, when the detected condition 216 and 226 is below the threshold, but near the threshold, this signifies that that condition of the user is not above the threshold but is still not a desired condition. When the condition is near or at the threshold, such as when the threshold is five and the condition is a 4 or 5, the current application executing on the processor 206 may initiate a command 218/230 to micro-modify the compartment 208. A micro-modification may be a portion of a normal modification. For example, the compartment 208 may be adjusted a small amount, such as a 5% increase or decrease. The current application determines the outcome of the micro-modification and whether the change lowered or raised the user's condition, and future modifications are adjusted according to the determined outcome. The modification and/or the micro-modification 220 and 232 should not be a modification to the compartment 208 to awake or cause a change to the current sleeping pattern of the user 210.

In one embodiment, the determined current condition 226 after the modification of the compartment 208 analyzes whether the condition of the user 210 is heading further above the threshold or heading further below the threshold. When the condition is heading further below the threshold, then modifications to the compartment 208 (either modifications or micro-modifications) are made more often in an attempt to raise the condition of the user 210. The current application may apply more modifications 232 to the compartment 208, wherein each modification is a micro-modification that, in time, equals a complete modification. These micro-modifications are made over a period of time such that the modifications do not bother the user 210 (for example, seconds or minutes, or a modification every 10 seconds). For example, the sensor receives data of the user 222, which is sent to the processor 206, wherein the current application executing on the processor 206 examines one or more of the blood flow, breathing, heart rate, the movement of the chest, the sounds of distress including snoring, gasping for air, coughing and the like.

In one embodiment, the modification may include playing white noise/music from the processor where speakers on or near the surface 204 present the sound. The music is at a volume that does not bother the user 210 and may increase in volume as the condition of the user 210 drops either below the threshold or further below the threshold. In another embodiment, the sound may be played through the said speakers to mask the modifications 220 and 232 to the compartment 208. For example, if the user 210 is getting worse, such as going from a level of 6 to 8, this is an alarming trend. The system initiates micro-modifications, which may make noise during the modification. The system turns on white noise at a volume level of 1. At a later time, the system rechecks the condition, and if the condition has worsened, such as going from a level 8 to 11 (the trend continuing), the system raises the volume to level 2. When the condition of the user 210 begins to trend downward, the system changes the sound to be at volume level 1.

In one embodiment, when the condition of the user is below the threshold 228, the current setting of at least one compartment 208 and the currently examined condition are stored. When the same condition is experienced at a later time, such as when data from the sensor 202 is analyzed by the current application executing on the processor 206, the current application issues modify commands 218/230 in the form of micro-modifications (as further detailed herein) to the at least one compartment 208 over a period of time such that the at least one compartment 208 is set to the previous, stored setting. For example, when the user snores, the current application detects the condition and returns the setting (through micro-modifications every 10 seconds) to bring at least one compartment 208 to the same setting that alleviated the snoring at a previous time.

In one embodiment, the current application, executing on the processor 206, determines the health-related characteristics of the user 210. This may be determined when the sensor 202 sends data 214/224 to the processor 206 related to the user 210. When the data reflects a health-related condition, such as when the sensor 202 detects data related to gasping for air, a breathing problem, and the like. The current application may send the health-related characteristics to a device associated with the user 210. In another embodiment, when the health-related characteristics indicate that the characteristics are greater than a threshold, the system 200 may perform another action. The another action may be one or more of sending a notification to the device associated with the user to issue a sound (such as an alarm) from the device to attempt to wake the user. The another action may be the sending of a notification to a device associated with another entity, such as a server associated with emergency personnel (e.g., 911 call). This may occur when the alarm has sounded, and the user continues not to respond accordingly. In yet another embodiment, the device associated with the user may not be notified until a period, such as only after 10 μm. In yet another embodiment, the processor 206 plays the alarm using a speaker attached to the surface 204.

In one embodiment, the device associated with the user 210 continues to obtain health-related data during the day as the user 210 carries the device. When the device becomes proximate the surface 204, the obtained health-related data on the device is sent to the processor 206, wherein the current application analyzes the data and adjusts the compartments 208 accordingly. In another embodiment, the adjustment to the compartment 208 may occur before the user 210 comes in contact with the surface 204.

In one embodiment, the current application executing on a processor, such as on the processor 206, may predict when the user will interact with the surface and begin modifying the surface, such as by preparing the surface at the fixed setting 234. The processor may communicate with a device associated with the user 210, such as a mobile device. In one embodiment, the current application is made aware of the device's geographical coordinates. This awareness may be through the receipt of coordinates sent by the device. When the coordinates are proximate the surface 204, or the coordinates indicate that the device will be proximate the surface 204, the current application sends a command 218/230 to modify the compartment 208. The setting may be at a previous modified setting 234. For example, the current application determines that the device's coordinates are approaching the surface 204, the surface being a seat in a vehicle. The current application modifies the at least one compartment 208 of the surface 204 at a previously set level.

In one embodiment, the device associated with the user 210 sets a surface 204 to a previous setting when the surface differs from a previous surface. The device sends a modified command 218/230 to the surface. For example, when the user is at a hotel, the mattress differs from the home mattress. The device retains the setting of the home surface 204 and issues modify commands 218/230 to the surface at the hotel. Processor 206 communicates with the device associated with the user and stores in the device's memory the setting of the compartment 208. In other examples, the surface may be a mattress on another bed, a seat cushion in a vehicle, a seat cushion on a sofa, a seat cushion in a movie theater, and the like.

In one embodiment, when the condition of the user 210 is above the threshold by an amount, a severe condition may be present. For example, the current application may detect, by the sensor 202, that the user is gasping for air. This condition may be determined by audio and/or video received 214/224 at the processor 206 from the sensor 202. The current application may then begin recording the user. The recording may be via the sensor 202, for example. The current application sends the recording in a notification to a mobile device associated with the user 210. The notification may trigger the mobile device to play a sound, such as a loud alarm. The alarm is meant to wake the user 210, wherein the user can play the recording on the mobile device to determine what happened. In another embodiment, when the user does not wake after a period of time, as detected by the sensor 202, the current application may send a notification to another entity, such as emergency personnel. In one embodiment, the recording is related to the condition, such as the length of the recording is related to an amount that the condition is above the threshold. For example, if the threshold for the number of coughs is set to 5 in 2-minutes, if the system determines that there are 9 coughs within the 2-minutes time period, the recording is set to collect 5-minutes of data. If the number of coughs is 6, then the recording is set to collect 2-minutes of data.

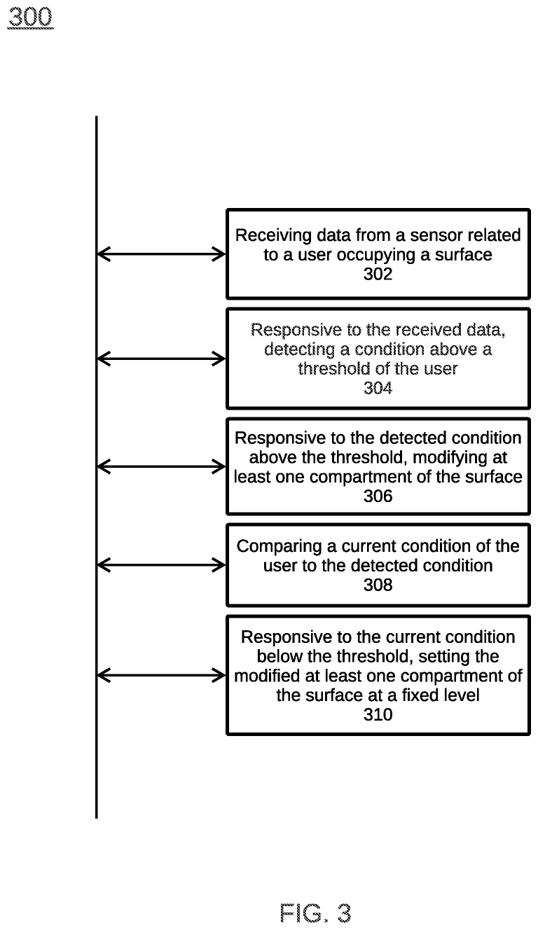

FIG. 3 illustrates a flow diagram 300, according to example embodiments. The solution includes one or more of receiving data from a sensor related to a user occupying a surface 302, responsive to the received data, detecting a condition above a threshold of the user 304, responsive to the detected condition above the threshold, modifying at least one compartment of the surface 306, comparing a current condition of the user to the detected condition 308, and responsive to the current condition below the threshold, setting the modified at least one compartment of the surface at a fixed level 310.

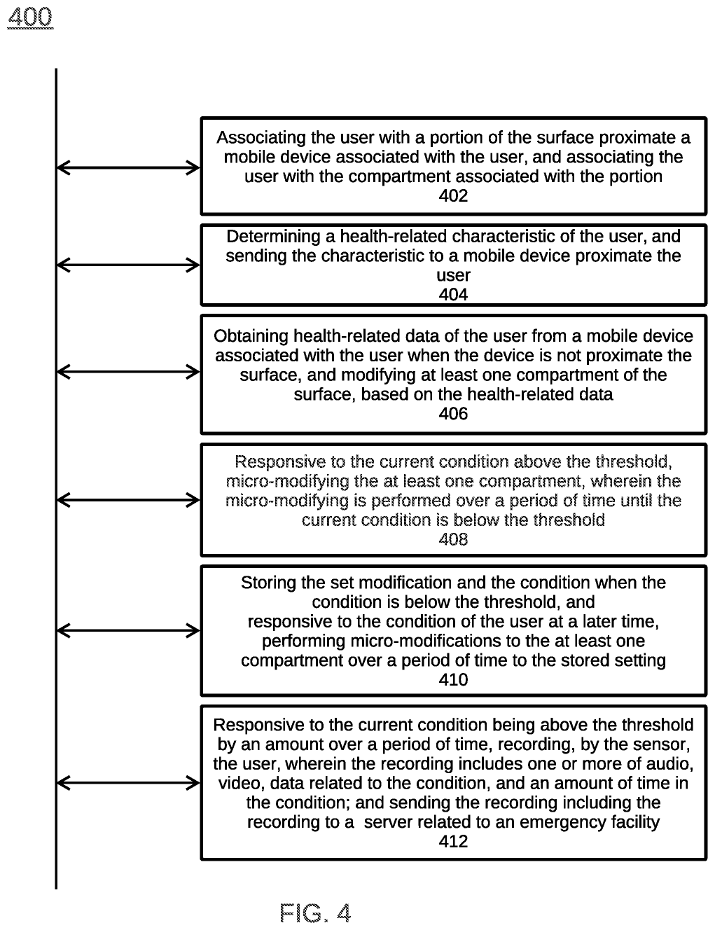

FIG. 4 illustrates another flow diagram 400, according to example embodiments. The solution includes one or more of associating the user with a portion of the surface proximate a mobile device associated with the user, and associating the user with the compartment associated with the portion 402, determining a health-related characteristic of the user, and sending the characteristic to a mobile device proximate the user 404, obtaining health-related data of the user from a mobile device associated with the user when the device is not proximate the surface, and modifying at least one compartment of the surface, based on the health-related data 406, responsive to the current condition above the threshold, micro-modifying the at least one compartment, wherein the micro-modifying is performed over a period of time until the current condition is below the threshold 408, storing the set modification and the condition when the condition is below the threshold, and responsive to the condition of the user at a later time, performing micro-modifications to the at least one compartment over a period of time to the stored setting 410, and responsive to the current condition being above the threshold by an amount over a period of time, recording, by the sensor, the user, wherein the recording includes one or more of audio, video, data related to the condition, and an amount of time in the condition, and sending the recording including the recording to a server related to an emergency facility 412. In one embodiment, data collected by a sensor 202/116 may be utilized to detect conditions of the user, such as through a determination of vital statistics of the user. The data collected by the sensor 202/116 may be sent to a communicably coupled processor, such as processor 206, where the instant solution executing on the processor 206 analyzes the received data from the sensor 202/116.

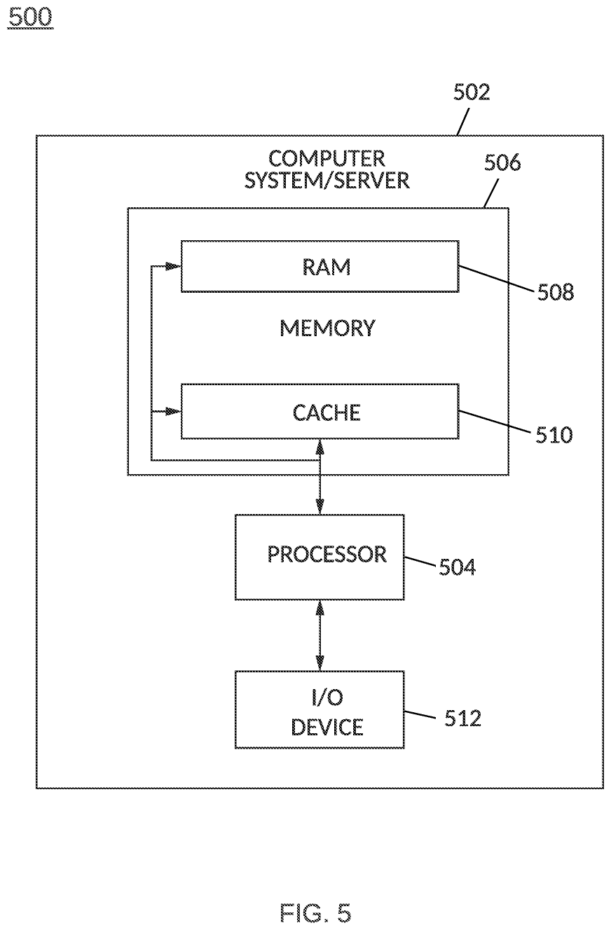

An exemplary storage medium may be coupled to the processor such that the processor may read information from, and write information to, the storage medium. In the alternative, the storage medium may be integral to the processor. The processor and the storage medium may reside in an application-specific integrated circuit (“ASIC”). In the alternative, the processor and the storage medium may reside as discrete components. For example, FIG. 5 illustrates an example computer system architecture 500, which may represent or be integrated into any of the above-described components, etc.

FIG. 5 is not intended to suggest any limitation as to the scope of use or functionality of embodiments of the application described herein. Regardless, the computing node 500 is capable of being implemented and/or performing any of the functionality set forth hereinabove.

In computing node 500, there is a computer system/server 502, operational with numerous other general or special purpose computing system environments or configurations. Examples of well-known computing systems, environments, and/or configurations that may be suitable for use with computer system/server 502 include, but are not limited to, personal computer systems, server computer systems, thin clients, thick clients, hand-held or laptop devices, multiprocessor systems, microprocessor-based systems, set-top boxes, programmable consumer electronics, network PCs, minicomputer systems, mainframe computer systems, and distributed cloud computing environments that include any of the above systems or devices, and the like.

Computer system/server 502 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 502 may be practiced in distributed cloud computing environments where tasks are performed by remote processing devices that are linked through a communications network. In a distributed cloud computing environment, program modules may be located in local and remote computer system storage media, including memory storage devices.

As shown in FIG. 5, the computer system/server 502 in cloud computing node 500 is shown in the form of a general-purpose computing device. The components of computer system/server 502 may include, but are not limited to, one or more processors or processing units 504, a system memory 506, and a bus that couples various system components, including system memory 506 to processor 504.

The bus represents one or more of several types of bus structures, including a memory bus or memory controller, a peripheral bus, an accelerated graphics port, and a processor or local bus using various bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 502 typically includes a variety of computer system readable media. Such media may be any available media accessible by computer system/server 502, including both volatile and non-volatile media, removable and non-removable media. System memory 506, in one example, implements the flow diagrams of the other figures. The system memory 506 can include computer system readable media in the form of volatile memory, such as random-access memory (RAM) 508 and/or cache memory 510. Computer system/server 502 may further include other removable/non-removable, volatile/non-volatile computer system storage media. By example only, memory 506 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”) and an optical disk drive for reading from or writing to a removable, non-volatile optical disk such as a CD-ROM, DVD-ROM or other optical media can be provided. In such instances, each can be connected to the bus by one or more data media interfaces. As will be further depicted and described herein, memory 506 may include at least one program product having a set (e.g., at least one) of program modules configured to carry out the functions of various embodiments of the application.

Program/utility, having a set (at least one) of program modules, may be stored in memory 506 by way of example, and not limitation, as well as an operating system, one or more application programs, other program modules, and program data. Each of the operating system, one or more application programs, other program modules, and program data or some combination thereof may include an implementation of a networking environment. Program modules generally carry out the functions and/or methodologies of various application embodiments described herein.

As will be appreciated by one skilled in the art, aspects of the present application may be embodied as a system, method, or computer program product. Accordingly, aspects of the present application may take the form of an entire hardware embodiment, an entire software embodiment (including firmware, resident software, micro-code, etc.), or an embodiment combining software and hardware aspects that may all generally be referred to herein as a “circuit,” “module” or “system.” Furthermore, aspects of the present application may take the form of a computer program product embodied in one or more computer readable medium(s) having computer readable program code embodied thereon.

Computer system/server 502 may also communicate with one or more external devices via an I/O device 512 (such as an I/O adapter), which may include a keyboard, a pointing device, a display, a voice recognition module, etc., one or more devices that enable a user to interact with computer system/server 502, and/or any devices (e.g., network card, modem, etc.) that enable computer system/server 502 to communicate with one or more other computing devices. Such communication can occur via I/O interfaces of device 512. Still yet, computer system/server 502 can communicate with one or more networks such as a local area network (LAN), a general wide area network (WAN), and/or a public network (e.g., the Internet) via a network adapter. As depicted, device 512 communicates with the other components of the computer system/server 502 via a bus. It should be understood that although not shown, other hardware and/or software components could be used in conjunction with computer system/server 502. Examples include, but are not limited to: microcode, device drivers, redundant processing units, external disk drive arrays, RAID systems, tape drives, data archival storage systems, etc.

Although an exemplary embodiment of at least one of a system, method, and non-transitory computer readable medium has been illustrated in the accompanying drawings and described in the foregoing detailed description, it will be understood that the application is not limited to the embodiments disclosed, but is capable of numerous rearrangements, modifications, and substitutions as set forth and defined by the following claims. For example, the system's capabilities of the various figures can be performed by one or more of the modules or components described herein or in a distributed architecture and may include a transmitter, receiver, or pair of both. For example, all or part of the functionality performed by the individual modules may be performed by one or more of these modules. Further, the functionality described herein may be performed at various times and in relation to various events, internal or external to the modules or components. Also, the information sent between various modules can be sent between the modules via at least one of a data network, the Internet, a voice network, an Internet Protocol network, a wireless device, a wired device, and/or via plurality of protocols. Also, the messages sent or received by any modules may be sent or received directly and/or via one or more of the other modules.

One skilled in the art will appreciate that a “system” could be embodied as a personal computer, a server, a console, a personal digital assistant (PDA), a cell phone, a tablet computing device, a smartphone, or any other suitable computing device, or combination of devices. Presenting the above-described functions as being performed by a “system” is not intended to limit the scope of the present application in any way but is intended to provide one example of many embodiments. Indeed, methods, systems, and apparatuses disclosed herein may be implemented in localized and distributed forms consistent with computing technology.

It should be noted that some of the system features described in this specification have been presented as modules to more particularly emphasize their implementation independence. For example, a module may be implemented as a hardware circuit comprising custom very large-scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together but may comprise disparate instructions stored in different locations that, when joined logically together, comprise the module and achieve the stated purpose for the module. Further, modules may be stored on a computer-readable medium, such as a hard disk drive, flash device, random access memory (RAM), tape, or any other such medium used to store data.

Indeed, a module of executable code could be a single instruction or many instructions and may even be distributed over several code segments, among different programs, and across several memory devices. Similarly, operational data may be identified and illustrated within modules, embodied in any suitable form, and organized within any suitable type of data structure. The operational data may be collected as a single data set or distributed over different locations, including over different storage devices, and may exist, at least partially, merely as electronic signals on a system or network.

It will be readily understood that the components of the application, as generally described and illustrated in the figures herein, may be arranged and designed in a wide variety of different configurations. Thus, the detailed description of the embodiments is not intended to limit the scope of the application as claimed but is merely representative of selected embodiments.

One with ordinary skill in the art will readily understand that the above may be practiced with steps in a different order and/or hardware elements in configurations that are different from those disclosed. Therefore, although the application has been described based upon these preferred embodiments, it would be apparent to those of skill in the art that certain modifications, variations, and alternative constructions would be apparent.

While preferred embodiments of the present application have been described, it is to be understood that the embodiments described are illustrative only, and the scope of the application is to be defined solely by the appended claims when considered with a full range of equivalents and modifications (e.g., protocols, hardware devices, software platforms, etc.) thereto.

In one embodiment, the modification of the at least one compartment increases blood flow to at least one portion of the user and/or better aligns the user occupying the surface such that a sleeping position of the user allows for a correct posture. The modification may also increase oxygen flow for the user and/or decrease noise emanating from the user, such as coughing or snoring.

What is claimed:

1. A method, comprising:
receiving data from a sensor related to a user occupying a surface;
responsive to the received data, detecting a condition above a threshold of the user;
responsive to the detected condition being above the threshold, modifying at least one compartment of the surface;
comparing a current condition of the user to the detected condition; and
responsive to the current condition being below the threshold, setting the modified at least one compartment of the surface at a fixed level.

2. The method of claim 1, comprising:
associating the user with a portion of the surface proximate a mobile device associated with the user; and
associating the user with the compartment associated with the portion.

3. The method of claim 1, comprising
determining a health-related characteristic of the user based on the comparing of the user; and
sending the characteristic to a mobile device proximate the user.

4. The method of claim 1, comprising:
obtaining health-related data of the user from a mobile device associated with the user when the device is not proximate the surface; and
modifying at least one compartment of the surface, based on the health-related data.

5. The method of claim 1, comprising, responsive to the current condition above the threshold, micro-modifying the at least one compartment, wherein the micro-modifying is performed over a period of time until the current condition is below the threshold.

6. The method of claim 1, comprising:
storing the set modification and the condition when the condition is below the threshold; and
responsive to the condition of the user at a later time, performing micro-modifications to the at least one compartment over a period of time to the stored setting.

7. The method of claim 1, comprising:
responsive to the current condition being above the threshold by an amount over a period of time, recording, by the sensor, the user, wherein the recording includes one or more of audio, video, data related to the condition, and an amount of time in the condition; and
sending the recording including the recording to a server related to an emergency facility.

8. A system, comprising:
a processor; and
a memory, wherein the processor and the memory are communicably coupled,
wherein the processor is configured to:
receive data from a sensor related to a user that occupies a surface;
responsive to the received data, detect a condition above a threshold of the user;
responsive to the detected condition is above the threshold, modify at least one compartment of the surface;
compare a current condition of the user to the detected condition; and
responsive to the current condition is below the threshold, set the modified at least one compartment of the surface at a fixed level.

9. The system of claim 8, wherein the processor is configured to:
associate the user with a portion of the surface proximate a mobile device associated with the user; and
associate the user with the compartment associated with the portion.

10. The system of claim 8, wherein the processor is configured to:
determine a health-related characteristic of the user; and
send the characteristic to a mobile device proximate the user.

11. The system of claim 8, wherein the processor is configured to:
obtain health-related data of the user from a mobile device associated with the user when the device is not proximate the surface; and
modify at least one compartment of the surface, based on the health-related data.

12. The system of claim 8, wherein the processor is configured to, responsive to the current condition above the threshold, micro-modify the at least one compartment, wherein the micro-modify is performed over a period of time until the current condition is below the threshold.

13. The system of claim 8, wherein the processor is configured to:
store the set modification and the condition when the condition is below the threshold; and
responsive to the condition of the user at a later time, perform micro-modifications to the at least one compartment over a period of time to the stored setting.

14. The system of claim 8, wherein the processor is configured to:
responsive to the current condition being above the threshold by an amount over a period of time, record, by the sensor, the user, wherein the recording includes one or more of audio, video, data related to the condition, and an amount of time in the condition; and
send the recording that includes the recording to a server related to an emergency facility.

15. A computer readable storage medium comprising instructions, that when read by a processor, cause the processor to perform:
receiving data from a sensor related to a user occupying a surface;
responsive to the received data, detecting a condition above a threshold of the user;
responsive to the detected condition being above the threshold, modifying at least one compartment of the surface;
comparing a current condition of the user to the detected condition; and
responsive to the current condition being below the threshold, setting the modified at least one compartment of the surface at a fixed level.

16. The computer readable storage medium of claim 15, wherein the instructions cause the processor to perform:
associating the user with a portion of the surface proximate a mobile device associated with the user; and
associating the user with the compartment associated with the portion.

17. The computer readable storage medium of claim 15, wherein the instructions cause the processor to perform:
determining a health-related characteristic of the user; and
sending the characteristic to a mobile device proximate the user.

18. The computer readable storage medium of claim 15, wherein the instructions cause the processor to perform:
obtaining health-related data of the user from a mobile device associated with the user when the device is not proximate the surface; and
modifying at least one compartment of the surface, based on the health-related data.

19. The computer readable storage medium of claim 15, wherein the instructions cause the processor to perform, responsive to the current condition above the threshold, micro-modifying the at least one compartment, wherein the micro-modifying is performed over a period of time until the current condition is below the threshold.

20. The computer readable storage medium of claim 15, wherein the instructions cause the processor to perform:
storing the set modification and the condition when the condition is below the threshold; and
responsive to the condition of the user at a later time, performing micro-modifications to the at least one compartment over a period of time to the stored setting.
